(19) United States
(12) Patent Application Publication
(10) Pub. No.: US 2019/0060924 A1
(43) Pub. Date: Feb. 28, 2019

(54) SYSTEM AND NOZZLE APPARATUS FOR ELECTROSTATIC SPRAYING

(71) Applicant: ELECTROSTATIC SPRAYING SYSTEMS, INC., WATKINSVILLE, GA (US)

(72) Inventors: RICHARD BRUCE WHITING, WATKINSVILLE, GA (US); CAMERON D. HOBBS, WATKINSVILLE, GA (US); JOEL S. HAYES, MADISON, GA (US)

(21) Appl. No.: 16/104,328

(22) Filed: Sep. 6, 2018

Related U.S. Application Data
(63) Continuation of application No. 16/104,339, filed on Aug. 17, 2018.
(60) Provisional application No. 62/549,589, filed on Aug. 24, 2017.
(60) Provisional application No. 62/549,598, filed on Aug. 24, 2017.

(51) Int. Cl. B05B 5/043

(52) U.S. Cl. CPC B05B 5/043

(57) ABSTRACT

A liquid is mixed with a pressurized air flow through an orifice which breaks it into particles. The liquid particles are entrained in the liquid flow and come into contact with an energized component in the form of a cone or frustum that is in contact with an electrode. The energized component defines a mixing chamber within a spray nozzle. As a result, while in the mixing chamber, the particles become electrostatically charged before exiting the spray nozzle. Embodiments also include a removable cap component within which the energized component nests. The cap component may include a pair of windows that cooperate with tabs on the body of the spray nozzle in order to mechanically engage the cap to the body such that the energized component is properly positioned to mate with the electrode and define the mixing chamber.

### BACKGROUND

The present disclosure relates to electrostatic spraying solutions and, more particularly, to one or both of a novel system and nozzle apparatus for atomizing a liquid, such as a disinfectant, applying an electrostatic charge to the atomized liquid and then spraying the charged particles of the atomized liquid into the atmosphere. The need for efficient and effective disinfectant systems and methods is prevalent in the healthcare industry and travel industry, among others. The ability to quickly and effectively disinfect a hospital room, or a nursing home dormitory, or a cruise ship cabin is readily evident.

Electrostatic spraying systems known in the art either require manual operation or are incapable of automatically spraying an entire space, such as a room, without a human operator. Additionally, electrostatic spraying nozzle apparatuses known in the art are difficult to repair, prone to fouling, inconsistent in application of electrical charge, and difficult to calibrate such that a consistent atomization of a liquid is achieved.

Therefore, there is a need in the art for a 360-degree electrostatic spray cart that overcomes the deficiencies in the prior art relative to automated electrostatic spraying. Further, there is a need in the art for a new and improved electrostatic spray nozzle that overcomes the deficiencies in the prior art relative to electrostatic spray nozzles.

### SUMMARY

An exemplary induction electrostatic spraying nozzle according to the solution includes connections to operably couple to each of an electric power supply, a fluid chemical supply, and a compressed air supply. Further, the electrostatic spray nozzle includes an electrostatic charge component operably coupled to an electrode that energizes the electrostatic charge component. The electrostatic charge component defines a mixing chamber within the automated electrostatic spray nozzle. Actuation of the electrostatic spray nozzle causes atomization of a fluid flow from the fluid chemical supply, electrostatic charging of the atomized fluid flow, and discharging of the electrostatically charged atomized fluid flow from the electrostatic spray nozzle.

The electrostatic charge component that defines the mixing chamber within the electrostatic spray nozzle may be in the general shape or form of a frustum. Further, the electrostatic spray nozzle may include a removable cap component. The electrostatic charge component may be integrated within the removable cap component or, alternatively, may be separable from the removable cap component. Also, the electrostatic spray nozzle may include a body that includes a pair of locking tabs and a removable cap component that includes a complimentary pair of locking windows such that the removable cap component is operable to mechanically engage with the body when the locking windows receive the locking tabs.

### BRIEF DESCRIPTION OF THE DRAWINGS

FIG. 1 is an illustration of an exemplary application of an electrostatic spraying system according to an embodiment of the solution;

FIG. 2 is an illustration of an exemplary embodiment of an electrostatic spraying system according to the solution, shown in a cart form;

FIG. 3 is a functional block diagram illustrating an interior view of the cabinet of the electrostatic spraying system embodiment of FIG. 2;

FIG. 4 is a close-up view of the exemplary gearing arrangement and 360-degree rotary union of the electrostatic spraying system embodiment of FIG. 2;

FIGS. 5A and 5B collectively illustrate an improved electrostatic spray nozzle according to an embodiment of the solution, shown assembled;

FIG. 6 is an illustration of the embodiment of an improved electrostatic spray nozzle of

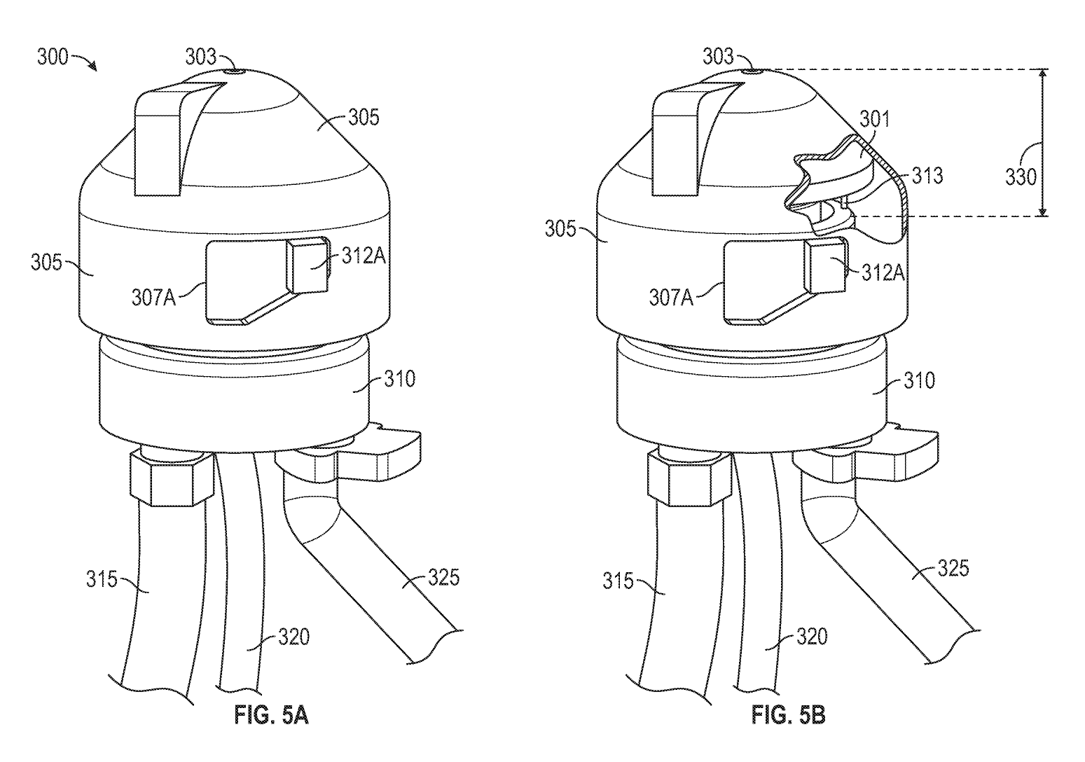

FIGS. 5A and 5B, shown with the nozzle cap disengaged from the nozzle body; and

FIG. 7 is a sectioned view of the electrostatic charge cone and nozzle cap arrangement of the improved electrostatic spray nozzle of FIGS. 5A and 5B.

### DETAILED DESCRIPTION

Various embodiments, aspects and features of the present solution encompass either or both of an improved electrostatic spray nozzle and an improved electrostatic spraying system configured to be placed in an area, such as a room, and automatically spray in a 360-degree manner in a continuous rotation. As would be understood by one of ordinary skill in the art of electrostatic spraying, an electric charge may be applied to, or induced on, an atomized flow of chemical such that charged droplets of the chemical are electrically attracted to surfaces that may harbor pathogens or the like.

Embodiments of the solution in the form of a spray nozzle are advantageous over prior art spray nozzles for at least the reason that they may comprise an electrostatic charge cone positioned within a mixing chamber such that a high percentage of particles in an atomized liquid flow come into contact with the electrostatic charge cone prior to exiting the spray nozzle. By coming into contact with the electrostatic charge cone, an electric charge is efficiently induced to the particles. Also advantageously, embodiments of the solution in the form of a spray nozzle may comprise a nozzle cap configured to consistently engage mechanically with the body of the spray nozzle such that a mixing chamber sized in view of Boyle's law and other physical considerations is defined within the nozzle in a dimensionally consistent manner.

Embodiments of the solution in the form of an inductive electrostatic spraying system are advantageous over prior art systems for at least the reason that they may be used without manual operation and without need for repositioning to completely spray a target space. Notably, embodiments of an electrostatic spraying system according to the solution may or may not comprise an electrostatic spraying nozzle that includes an electrostatic charge cone positioned within a mixing chamber such that a high percentage of particles in an atomized liquid flow come into contact with the electrostatic charge cone prior to exiting the spray nozzle. Moreover, embodiments of an electrostatic spraying system according to the solution may or may not comprise an electrostatic spraying nozzle that includes a nozzle cap configured to consistently engage mechanically with the body of the spray nozzle such that a mixing chamber sized in view of Boyle's law and other physical considerations is defined within the nozzle in a dimensionally consistent manner.

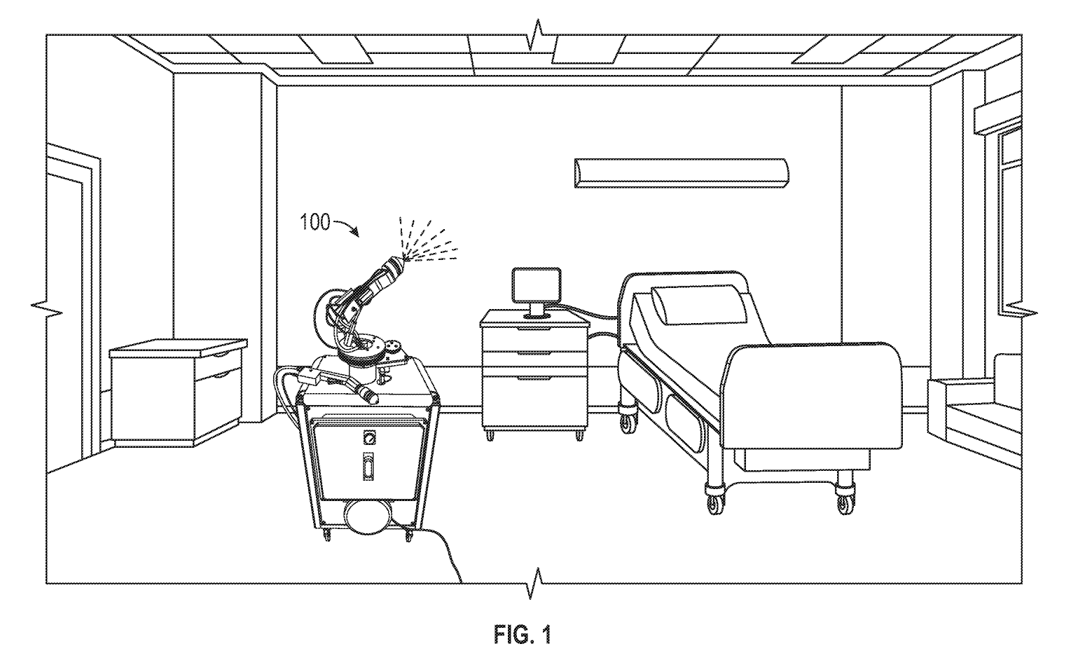

Turning now to the figures, FIG. 1 illustrates an exemplary application of an electrostatic spraying system 100 according to an embodiment of the solution. As can be understood from the FIG. 1 illustration, the electrostatic spraying system 100 is positioned substantially in the center of a room and has been activated. The system 100 is electrically coupled to a wall outlet in order that its compressor (not seen in the FIG. 1 illustration) and other components may be powered. The system operates according to an executable software program that dictates a pattern of rotation for the electrostatic spray nozzle.

As shown in more detail in subsequent figures, the electrostatic spray nozzle may be coupled to a rotary union such that the nozzle may be continuously rotated in a 360-degree pattern. Depending on embodiment and the executable program used to govern the spray pattern, the electrostatic spray nozzle may be rotated continuously in one direction for a duration of time (e.g., clockwise), or continuously in one direction for a first duration of time (e.g., clockwise) followed by continuously in a second direction for a second duration of time (e.g. counterclockwise). The electrostatic spray nozzle may also be configured to translate from an uppermost direction to a lower most direction (i.e., “up and down”) as it rotates. The electrostatic spray nozzle may also be held in a certain position for a relatively longer period of time than it is held in other positions, as may be dictated by the executable program. In these ways, an embodiment of the system 100 may be used to deliver a high degree of efficacy when applying a disinfectant to surfaces in a target space.

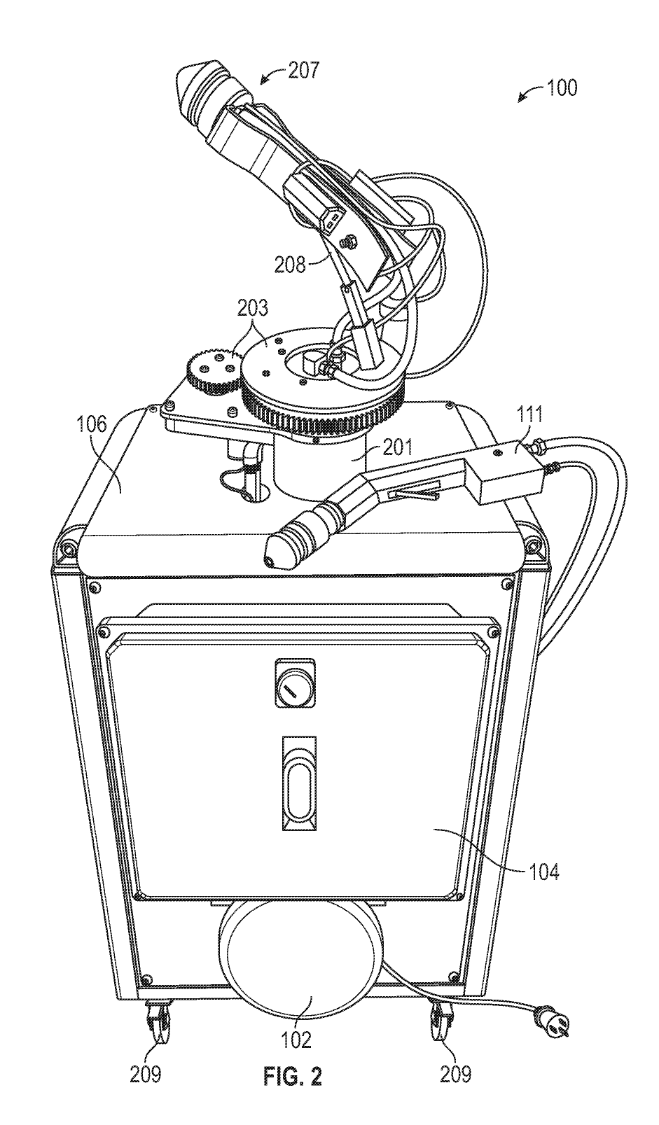

FIG. 2 illustrates an exemplary embodiment of an electrostatic spraying system 100 according to the solution, shown in a cart form. It is an advantage of the novel solution that the system 100 may be positioned centrally in a room such that it may automatically distribute electrostatically charged spray around the entire room without having to be repositioned. As explained above, the automated electrostatic nozzle 207 may be configured via integration to a rotary union 201 and gear box 203 such that it may rotate continuously as it is spraying. Moreover, the automated electrostatic nozzle 207 may be configured via integration to a linear actuator 208 such that it can translate vertically “up and down” as it rotates, thereby increasing the efficacy of its spray coverage to the target space and surfaces.

Additionally, embodiments of the system 100 may include a manual electrostatic spray gun 111 in addition to the automated electrostatic nozzle 207. Advantageously, the manual electrostatic spray gun 111 may be useful for an operator of the system 100 to manually apply electrostatically charged chemical spray to target surfaces within the space and/or to ensure that electrostatically charged spray is applied to hard to reach or critical areas within the space. The manual electrostatic spray gun 111 may be mounted on the exterior of the cabinet 106 or stored in its interior.

As can be seen in the FIG. 1 illustration, the system 100 may include casters 209 such that it may be easily moved and positioned in a target space. Once positioned, the cart may be electrically coupled to a power source, such as a 110V or 120V wall socket accessible power source, via a retractable power cord 102. With power supplied to the system 100, an on/off switch of a control panel 104 may be used to “start” the system 100 so that it automatically distributes the electrostatically charged spray, as will be described in more detail below.

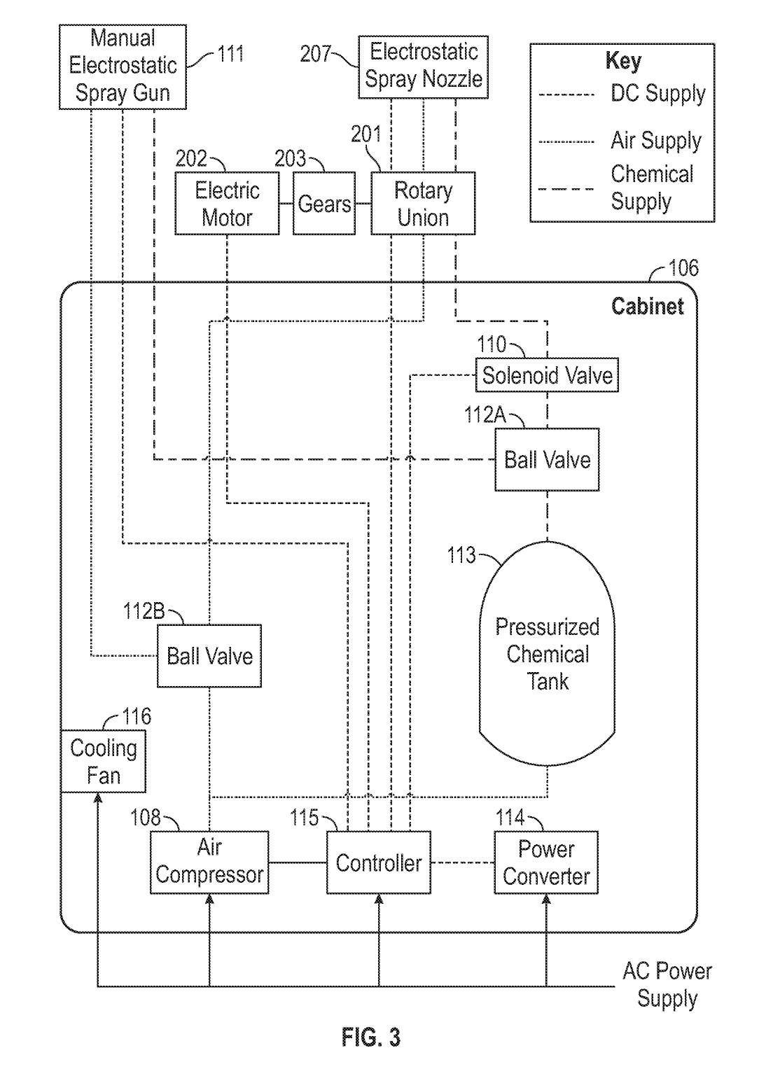

FIG. 3 is a functional block diagram illustrating an interior view of the cabinet 106 of the electrostatic spraying system 100 embodiment of FIG. 2. As can be seen in the FIG. 3 illustration, a power source (such as a 120Vac power source via power cord 102) supplies power to one or more components of the system 100, namely, a power converter 114, a controller 115, and air compressor 108 and a cooling fan 116. The power converter 114 converts the alternating current power supply to a direct current power supply, as would be understood by one of ordinary skill in the art. The cooling fan 116 is configured to move air through the cabinet 106 in order to cool the interior of the cabinet 106.

The air compressor 108 supplies compressed air to either the electrostatic spray nozzle 207 or the manual electrostatic spray gun 111. A valve 112B, such as but not limited to a three-way ball valve, diverts the compressed air to either of the electrostatic spray nozzle 207 or the manual electrostatic spray gun 111, as would be understood by one of ordinary skill in the art of valves. Similarly, valve 112A diverts chemical from pressurized chemical tank 113 to either of the electrostatic spray nozzle 207 or the manual electrostatic spray gun 111. It is envisioned that the valve 112B (as well as valve 112A) may be either manually operated or automated. In the FIG. 3 illustration, the valves 112 are manually operated. However, if either or both of the valves 112 were automated, the controller 115 may be configured to actuate the valve(s) 112.

The air compressor 108 may also supply compressed air to the pressurized chemical tank 113 in order to pressurize the chemical for delivery to nozzle 207 or spray gun 111. Notably, although the exemplary embodiment of system 100 illustrated in FIG. 3 depicts pressurized chemical tank 113 receiving its motive force from air compressor 108, it is envisioned that other embodiments may simply employ a previously pressurized chemical tank. A solenoid valve 110 may be positioned downstream of valve 112A so that pressurized chemical from tank 113 may be isolated when system 100 is not in use, thereby preventing leakage of the chemical through nozzle 207 or spray gun 111. The solenoid valve is actuated by virtue of receiving a DC power supply from controller 115, as would be understood by one of ordinary skill in the art. The solenoid valve 110 may be a “normally closed” arrangement or a “normally open” arrangement depending on the embodiment of the solution. As previously mentioned, the solenoid valve 110 may work to prevent or mitigate “leak by” due to residual system pressure when the system 100 is turned off.

The power source converter 114 supplies DC electric power to the controller that, in turn, and according to a preprogrammed executable logic, supplies DC power to one or more of the solenoid 110, electrostatic spray nozzle 207, motor 202 and manual electrostatic spray gun 111. As can be understood from the FIG. 3 illustration, the pressurized chemical and compressed air, as well as the DC electrical power supply, are supplied through the rotary union 201 to the electrostatic spray nozzle 207. Advantageously, the rotary union 201 allows for the liquid chemical supply, the compressed air supply, and the electrical power supply to be provided to the electrostatic spray nozzle 207 while also providing a means by which the electrostatic spray nozzle 207 may be continuously rotated and translated vertically as described above. Similarly, the pressurized chemical and compressed air, as well as the DC electrical power supply, are supplied to the manual electrostatic spray gun 111.

Returning to the operation of the electrostatic spray nozzle 207, the controller supplies the power supply to an electric motor 202 and a linear actuator 208 (not shown in the FIG. 3 illustration). The electric motor 202 drives a gear arrangement 203 that operates to actuate a turntable function of the rotary union 201, thereby providing for the continuous circular rotation of the electrostatic spray nozzle 207 when in operation. Similarly, the linear actuator 208 operates to actuate a hinged mechanism such that the electrostatic spray nozzle 207 is translated up and down vertically or positioned at a certain desirable angle, as the case may be and according to the programmable instructions executed by controller 115.

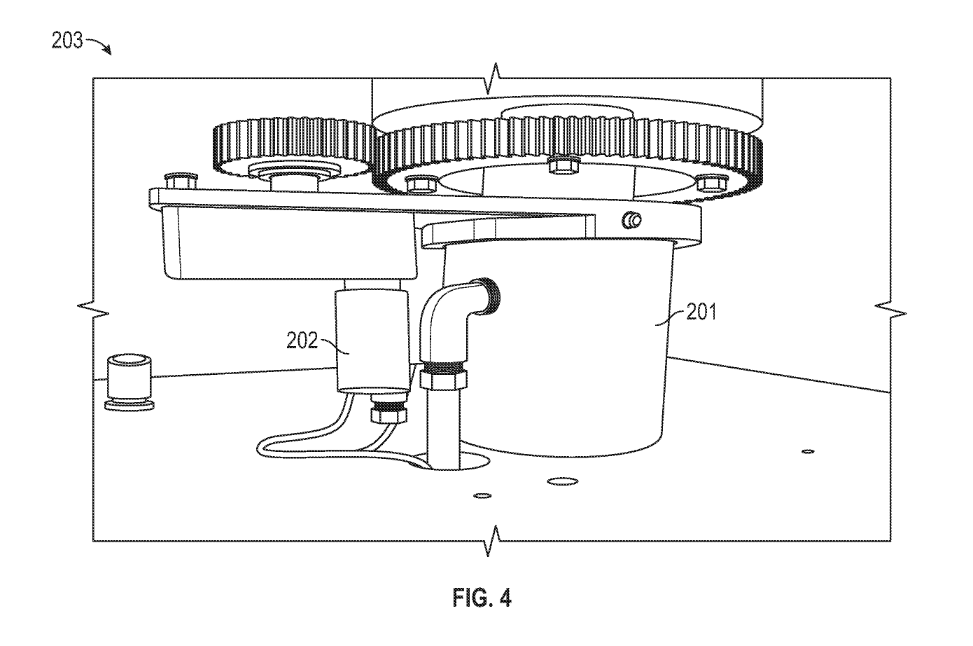

FIG. 4 is a close-up view of the exemplary gearing arrangement 203 and 360-degree rotary union 201 of the electrostatic spraying system 100 embodiment of FIG. 2. The controller 115 provides a DC power supply to both the motor 202 (as understood from the FIG. 3 illustration) and the electrostatic spray nozzle 207 (by and through the 360-degree rotary union 201). As previously described, the motor 202 is configured to turn the gearing 203 that, in turn, may rotate the spray nozzle 207 in a continuous 360-degree rotation. The power supply wires that power the electrostatic nozzle 207 and a piston 208 configured to raise/lower the nozzle 207 supply power by and through the rotary coupling arrangement 201. The rotary union 201 includes a pair of “slip rings” that mate in such a way that electrical power may be transmitted while the coupling rotates. The rotary union 201 also accommodates the air supply lines and the liquid supply line, as can be understood from the FIG. 3 illustration.

As will become more clearly understood from the following figures, the electrostatic spray nozzle 207 (as well as the manual electrostatic spray gun 111) includes an internal mixing chamber wherein atomized liquid may come in contact with an energized electrode such that the atomized liquid becomes electrostatically charged. The compressed air lines, liquid supply lines and electrical supply wires, supply both the electrostatic spray nozzle 207 and manual electrostatic spray gun 111. With regards to the nozzle 107, the power supply provides electrical power for powering the tilt piston and internal electrode of the nozzle. The gearing 203 works to rotate the spray nozzle 207 in a continuous circular path. Depending on embodiment, the piston may be used to translate the spray nozzle 207 up and down while the gears 203 work to rotate the entire nozzle 207 along the aforementioned circular path (or some variation of the circular path). Alternatively, the piston 208 may be used to position the spray nozzle 207 at a fixed angle while the entire nozzle 207 is rotated. In these ways, embodiments of the solution may apply an electrostatically charged atomized liquid around and throughout an entire room without need for repositioning or translating back and forth along an arc less than a full circle.

Turning now to FIGS. 5 through 7, an exemplary embodiment of an improved electrostatic spray nozzle according to the solution will be shown and described. As mentioned above, embodiments of the solution in the form of system 100 may, or may not, include an electrostatic spray nozzle such that which is shown and described below. Even so, it is envisioned that nozzle 207 and/or manual spray gun 111 may comprise an electrostatic spray nozzle within the scope of the spray nozzle disclosed herein, although such is not a requirement.

FIGS. 5A and 5B illustrate an improved electrostatic spray nozzle 300 according to an embodiment of the solution, shown assembled. From the illustrations, it can be seen that a power supply wire 325, a liquid supply line 320 and a compressed air supply line 315 enter a body 310 of the spray nozzle 300. A nozzle cap 305 is mechanically engaged with the body 310. The means by which the nozzle cap 305 mechanically engages with the body 310 provides for a consistently dimensioned mixing chamber within the improved electrostatic spray nozzle 300 regardless of how many times the nozzle cap 305 may be disengaged and/or reengaged from/to the nozzle body 310.

As can be seen in the illustrations, the nozzle cap 305 includes an engagement window 307A configured and positioned to mechanically interface with a locking tab 312A protruding from the spray nozzle body 310. A complimentary engagement window 307B and locking tab 312B on the opposite side of the spray nozzle 300 cannot be seen in the FIGS. 5A and 5B views. The nozzle cap 305 may be quickly and easily removed from, and re-engaged to, the body 310 by way of “twisting” the nozzle cap 305 relative to the body 310. Advantageously, when the locking tabs 312 are engaged within the engagement windows 307, the nozzle cap 305 is precisely positioned relative to the body 310 such that a consistent dimension of an internal mixing chamber is achieved (such as may be generally represented by dimension 330 for example). Compared to prior art spray nozzles that use a threaded connection, for example, embodiments of the present solution provide a higher efficacy of atomization and electrostatic charging attributable to a consistent mixing chamber dimension. Further, because of the engagement window 307 and locking tab 312 arrangement explained above, embodiments of an improved electrostatic spray nozzle provide for quick and easy access to interior components of the spray nozzle for cleaning in the event of fouling.

Referring specifically to the FIG. 5B illustration, the cutaway portion of the nozzle cap 305 reveals the mechanical engagement of the electrode 313 to the electrostatic charge cone 301. Advantageously, when the locking tabs 312 are fully engaged into the respective locking windows 307, the nozzle cap 305 positions the electrostatic charge cone 301 such that its bottom surface touches the electrode 313 without crushing, bending or otherwise damaging the electrode 313. As aforementioned, the precisely dimensioned mixing chamber defined by the interior of the cone 313 (and above and around the atomizing orifice 317, not shown in the FIG. 5 illustrations) is formed when the cap 305 is engaged onto the body 310 while the electrode 113 contacts the cone 301. The electrode 313 electrically energizes the cone 301 so that a charge may be imparted to atomized liquid flow within the mixing chamber. Because the entire cone 301 is energized, thereby generating a relatively large charged surface area with which an atomized flow may come into contact, embodiments of the solution have improved efficacy of imparting charge to an atomized flow of liquid, over that of prior art solutions that simply rely on an electrode element protruded into the flow.

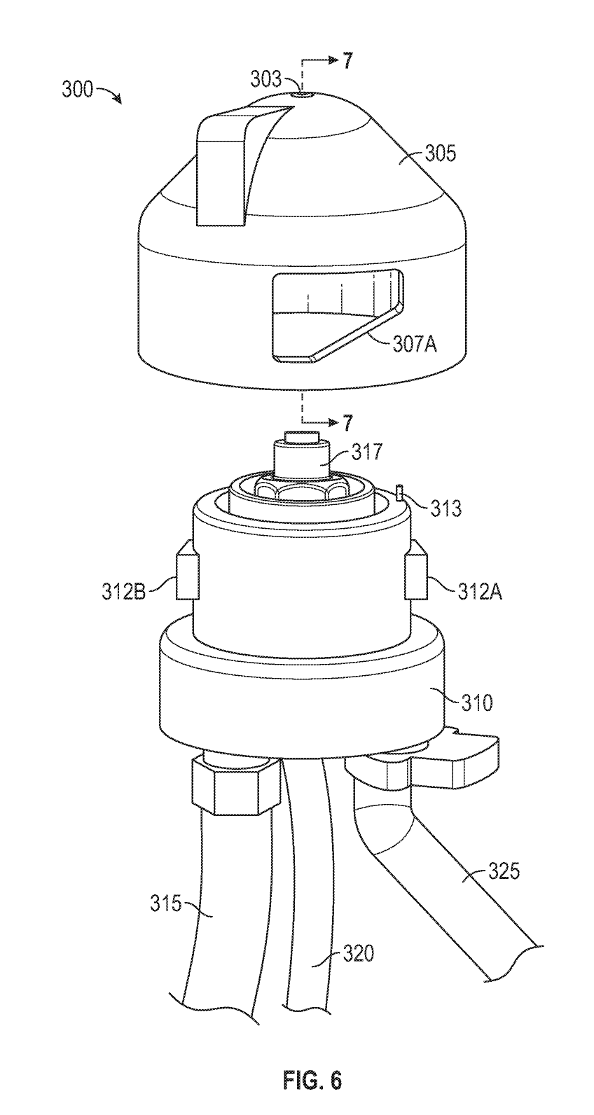

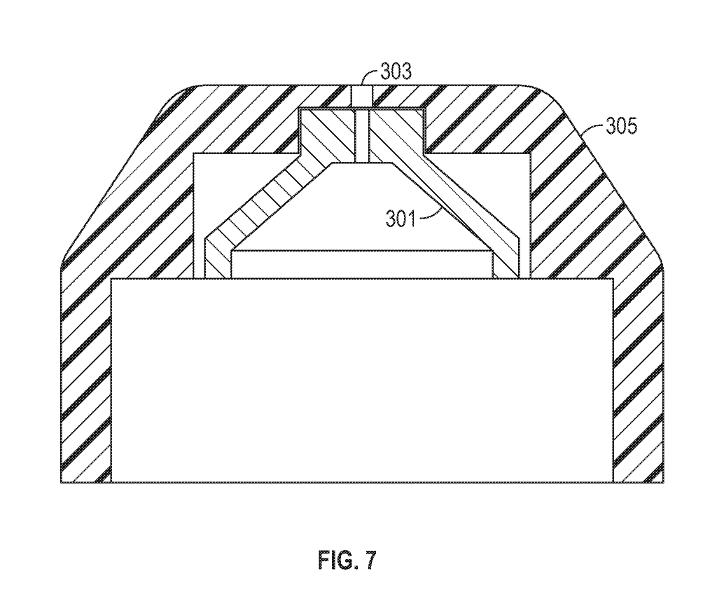

FIG. 6 illustrates the embodiment of an improved electrostatic spray nozzle 300 of FIGS. 5A and 5B, shown with the nozzle cap 305 disengaged from the nozzle body 310. As can be seen in the FIG. 3 illustration, an electrode 313 is exposed from the body 310 such that it engages with a contact surface 302 of an electrostatic charge cone 301 when the cap 305 is engaged to the body 310 (see FIG. 5B). Also, in FIG. 6 both the locking tabs 312A and 312B can be seen. As previously described, locking tabs 312A and 312B may be mechanically engaged with engagement windows 307 in the nozzle cap 305 such that an internal mixing chamber of a precise and preferred dimension is consistently defined every time the nozzle cap 305 is re-engaged (see FIG. 5B).

As would be understood by one of ordinary skill in the art of spray nozzles, an atomizing orifice 317 may be configured and positioned to atomize a liquid supply with a pressurized air supply (i.e., finely divide a liquid stream into a flow of divided liquid particles). The atomized flow, upon exiting the atomizing orifice 317, fills a mixing chamber defined within the interior cavity of the nozzle cap 305. While in the mixing chamber, the atomized flow is exposed to the electrostatic charge cone 301 before exiting the nozzle 300 through aperture 303. The electrostatic charge cone 301, having been electrically energized by virtue of its contact with electrode 313, imparts an electrostatic charge on the droplets that form the atomized flow.

FIG. 7 is a sectioned view of the electrostatic charge cone 301 and nozzle cap 305 arrangement of an improved electrostatic spray nozzle 300. As can be understood fully from the FIG. 7 illustration, the electrostatic charge cone 301 mechanically nests within the interior space of the nozzle cap 305. Both the electrostatic charge cone 301 and the nozzle cap 305 include holes that align to form aperture 303. As previously described, the interior or underside of the electrostatic charge cone 301, when it is nested in the nozzle cap 305 and the nozzle cap 305 is engaged to the body 310, forms a cavity space or mixing chamber within which an electrical charge may be imparted to droplets of an atomized chemical flow coming out of atomizing orifice 317 before its exit through aperture 303. Advantageously, because the electrode 313 energizes the entire electrostatic charge cone 301, the mixing chamber defined by the space beneath the charge cone 301 includes a relatively large charged surface area (essentially the entire interior/underside surface of the charge cone 301) with which to come into contact with an atomized mist or fog or flow emanating from the atomizing orifice 317. In this way, embodiments of the solution provide for a highly efficient electrostatic charging of the atomized flow.

It is envisioned that, in some embodiments of an improved electrostatic spray nozzle according to the solution, the electrostatic charge cone 301 may be permanently integrated into the nozzle cap; however, in other embodiments of an improved electrostatic spray nozzle according to the solution the electrostatic spray cone 301 may be separable from the nozzle cap in order to facilitate easy cleaning and replacement.

The present solution has been described using detailed descriptions of embodiments thereof that are provided by way of example and are not intended to limit the scope of the solution. The described embodiments comprise different features, not all of which are required in all embodiments of the solution. Some embodiments of the present solution utilize only some of the features or possible combinations of the features. Variations of embodiments of the present solution that are described, and embodiments of the present solution comprising different combinations of features noted in the described embodiments will occur to persons of the art. Moreover, it will be appreciated by persons skilled in the art that the present solution is not limited by what has been particularly shown and described herein above. Rather the scope of the invention is defined by the claims that follow.

What is claimed is:

1. An induction electrostatic spraying system, comprising:
a mobile support frame structure comprising casters;
an electric power source component;
a chemical reservoir;
an air compressor operably coupled to the electric power source component;
a controller operably coupled to the electric power source component; and
an automated electrostatic spray nozzle operably coupled via a rotary union to each of the electric power source component, chemical reservoir and air compressor, wherein the rotary union is operable for rotating the automated electrostatic spray nozzle in a continuous, circular pattern;
wherein actuation of the automated electrostatic spray nozzle by the controller causes atomization of a fluid flow from the chemical reservoir, electrostatic charging of the atomized fluid flow, and discharging of the electrostatically charged atomized fluid flow from the automated electrostatic spray nozzle.

2. The induction electrostatic spraying system of claim 1, wherein the mobile support frame structure comprises a closed cabinet defining an interior compartment.

3. The induction electrostatic spraying system of claim 2, wherein the interior compartment houses one or more of the electric power source component, chemical reservoir, air compressor, and controller.

4. The induction electrostatic spraying system of claim 2, further comprising one or more cooling fans operably coupled to the electric power source component and configured to cool the interior compartment.

5. The induction electrostatic spraying system of claim 1, wherein the electric power source component is configured to electrically couple to an alternating current power source and convert alternating current to direct current.

6. The induction electrostatic spraying system of claim 1, further comprising a manually operated electrostatic spray nozzle operably coupled to each of the electric power source component, chemical reservoir and air compressor, wherein actuation of the manually operated electrostatic spray nozzle by a user of the induction electrostatic spraying system causes atomization of a fluid flow from the chemical reservoir, electrostatic charging of the atomized fluid flow, and discharging of the electrostatically charged atomized fluid flow from the electrostatic spray nozzle.

7. The induction electrostatic spraying system of claim 6, further comprising a first valve for diverting a flow of chemical from the chemical reservoir and a second valve for diverting a compressed air supply from the compressor.

8. The induction electrostatic spraying system of claim 6, wherein the manually operated electrostatic spray nozzle comprises an electrostatic charge component operably coupled to an electrode that energizes the electrostatic charge component, wherein the electrostatic charge component defines a mixing chamber within the automated electrostatic spray nozzle.

9. The induction electrostatic spraying system of claim 8, wherein the electrostatic charge component is in the form of a frustum.

10. The induction electrostatic spraying system of claim 8, wherein the manually operated electrostatic spray nozzle comprises a removable cap component and the electrostatic charge component is integrated within the cap component.

11. The induction electrostatic spraying system of claim 8, wherein the manually operated electrostatic spray nozzle comprises a removable cap component and the electrostatic charge component is separable from the cap component.

12. The induction electrostatic spraying system of claim 8, wherein the manually operated electrostatic spray nozzle comprises a body and a removable cap component, wherein the body comprises a pair of locking tabs and the removable cap component comprises a pair of locking windows such that the removable cap component is operable to mechanically engage with the body when the locking windows receive the locking tabs.

13. The induction electrostatic spraying system of claim 1, wherein the automated electrostatic spray nozzle comprises an electrostatic charge component operably coupled to an electrode that energizes the electrostatic charge component, wherein the electrostatic charge component defines a mixing chamber within the automated electrostatic spray nozzle.

14. The induction electrostatic spraying system of claim 13, wherein the electrostatic charge component is in the form of a frustum.

15. The induction electrostatic spraying system of claim 13, wherein the automated electrostatic spray nozzle comprises a removable cap component and the electrostatic charge component is integrated within the cap component.

16. The induction electrostatic spraying system of claim 13, wherein the automated electrostatic spray nozzle comprises a removable cap component and the electrostatic charge component is separable from the cap component.

17. The induction electrostatic spraying system of claim 13, wherein the automated electrostatic spray nozzle comprises a body and a removable cap component, wherein the body comprises a pair of locking tabs and the removable cap component comprises a pair of locking windows such that the removable cap component is operable to mechanically engage with the body when the locking windows receive the locking tabs.

18. The induction electrostatic spraying system of claim 1, wherein the chemical reservoir is operably connected to the air compressor.

19. The induction electrostatic spraying system of claim 1, wherein the chemical reservoir is pre-pressurized.

20. The induction electrostatic spraying system of claim 1, wherein the automated electrostatic spray nozzle comprises a linear piston operable to raise and lower the automated electrostatic spray nozzle.
